(19) United States
(12) Patent Application Publication
(10) Pub. No.: US 2008/0026087 A1
(43) Pub. Date: Jan. 31, 2008

(54) Litchi Sinensis Extracts Containing Oligomeric Proanthocyanidins and Processes of Making Thereof

(76) Inventors: Santiago Rull Prous, Barcelona (ES); Small Alaoui Ismaili, Cerdanyola del Valles (ES); Bernd Fabry, Korschenbroich (DE)

Correspondence Address: COGNIS CORPORATION;PATENT DEPARTMENT, 300 BROOKSIDE AVENUE, AMBLER, PA, 19002, US

(21) Appl. No.: 10/561,698

(22) PCT Filed: Jun. 15, 2004
(86) PCT No.: PCT/EP04/06415

§ 371 Date: Mar. 22, 2007

(30) Foreign Application Priority Data
Jun. 24, 2003 (EP) 03014143.6

(51) Int. Cl. A61K 36/00; A61P 39/06; A61Q 19/00; C07D 311/30

(52) U.S. Cl. 424/777; 549/403

(57) ABSTRACT

A botanic extract of Litchi sinensis containing, based on active substance content, at least 15% by weight of an oligomeric proanthocyanidin of the OPC A2 type is disclosed. Processes of making such extracts containing at least 15% by weight of OPC A2 are also disclosed.

### FIELD OF THE INVENTION

This invention relates generally to food supplements and, more particularly to special botanic extracts with a high content of special active components, to a new process for their production and to their use in various fields.

### PRIOR ART

Extracts of the shells of plants of the genus Sapindaceae, particularly the species Litchi sinensis (Sonn.), are known for their high content of flavone derivatives, more particularly hydrogenation or oxidation products of 2-phenyl-4H-1-benzopyran or derivatives thereof, such as for example flavans, flavan-3-ols (catechols, catechol oligomers), flavan-3,4-diols (leucoanthocyanides), flavones, flavonols and flavonones. However, the extracts consist mainly of condensed tannins, so-called “oligomeric procyanodols” (OPCs). These are oligomers with 2 to 8 monomers of the catechol or epicatechol type, such as for example procyanidins, proanthocyanidins, procyanidoel, oligoprocyanidins, leucoanthocyanidins, leucodelphinins, leucocyanins and anthocyanogens. OPCs, more especially the particularly active proanthocyanidin A2 (OPC A2), exhibit properties similar to those of vitamin P, above all the inhibition of matrix metalloproteases (MMPs). However, MMPs have the property of attacking the dermal macromolecules of the connective tissue, such as proteoglycan, collagen, elastin, dissolving the peptide bonds and, hence, causally contributing to ageing of the skin. In the event of inflammatory processes in the skin, too, the macrophages and polymorphonuclear neutrophilic granulocytes release proteases, such as for example the serine protease elastase, or matrix metalloproteases (MMPs), such as collagenase, and another elastin-degrading elastase belonging to the MMPs.

In this connection, mention is made of European patent application EP 0978274 A1 (Kibun) which describes cosmetic preparations for topical application that contain emulsifiers of the sphingoglycolipid type together with litchi extracts, the litchi extracts being intended to depigment the skin. In addition, European patent application EP 0965328 A1 (Kao) discloses cosmetic preparations which contain litchi extracts together with special phosphoric acid esters. Neither of the documents in question contains any indication of how litchi extracts can be produced or any reference to their oral application as food supplements.

The production of litchi extracts has been found to be difficult in practice. Conventional extraction techniques using solvents of different polarity only give extracts with OPC A2 contents of up to 15% by weight—too little for extraction to be carried out economically and for the products to be marketed under economically reasonable conditions. Accordingly, the problem addressed by the present invention was to provide extracts of Litchi sinensis which would contain—based on the active substance—at least 15, preferably at least 20 and more particularly 20 to 25% by weight of oligomeric proanthocyanidins of the OPC A2 type, and a process for the production of such extracts. Another problem addressed by the present invention was to develop food supplements which, taken orally, would counteract both ageing of the skin and signs of inflammation.

### DESCRIPTION OF THE INVENTION

The present invention relates to botanic extracts containing—based on the active substance content—at least 15 and preferably 20 to 25% by weight of oligomeric proanthocyanidins of the OPC A2 type which can be obtained by
(a) subjecting shells of the fruit of Litchi sinensis to extraction with lower, optionally aqueous aliphatic alcohols,
(b) subjecting the extracts to chromatographic separation, optionally after concentration and/or filtration,
(c) subjecting the OPC A2-rich fraction obtained in the chromatography step to a liquid/liquid extraction and
(d) removing the resulting organic phase.

It has surprisingly been found that, by combining the process steps of “extraction”, “chromatography” and “liquid/liquid extraction”, it is now possible to obtain extracts which contain the OPC A2 in distinctly higher concentrations than known products and which are therefore far stronger in their MMP-inhibiting and anti-inflammatory effect.

The present invention also relates to a process for the production of botanic extracts containing—based on the active substance content—at least 15 and preferably 20 to 25% by weight of oligomeric proanthocyanidins of the OPC A2 type which comprises the steps of
(a) subjecting shells of the fruit of Litchi sinensis to extraction with lower, optionally aqueous aliphatic alcohols,
(b) subjecting the extracts to chromatographic separation, optionally after concentration and/or filtration,
(c) subjecting the OPC A2-rich fraction obtained in the chromatography step to a liquid/liquid extraction and
(d) removing the resulting organic phase.

### Extraction

The extracts may be prepared by methods known per se, i.e. for example by aqueous, alcoholic or aqueous/alcoholic extraction of the plants or parts thereof or shells of the litchi fruits. Suitable extraction processes are any conventional extraction processes, such as maceration, remaceration, digestion, agitation maceration, vortex extraction, ultrasonic extraction, countercurrent extraction, percolation, repercolation, evacolation (extraction under reduced pressure), diacolation and solid/liquid extraction under continuous reflux. Percolation is advantageous for industrial use. Litchi shells are preferably used as the starting material and may be mechanically size-reduced before the extraction process. Any size reduction methods known to the expert, for example freeze grinding, may be used. Preferred solvents for the extraction process are organic solvents, water (preferably hot water with a temperature above 80° C. and more particularly above 95° C.) or mixtures of organic solvents and water, more particularly low molecular weight alcohols with more or less high water contents. Extraction with methanol, ethanol and water-containing mixtures thereof is particularly preferred. The extraction process is generally carried out at 20 to 100° C. and preferably at 50 to 70° C. In one preferred embodiment, the extraction process is carried out in an inert gas atmosphere to avoid oxidation of the ingredients of the extract. This is particularly important where extraction is carried out at temperatures above 40° C. The extraction times are selected by the expert in dependence upon the starting material, the extraction process, the extraction temperature and the ratio of solvent to raw material, etc. After the extraction process, the crude extracts obtained may optionally be subjected to other typical steps, such as for example purification, concentration and/or decoloration. If desired, the extracts thus prepared may be subjected, for example, to the selective removal of individual unwanted ingredients. The extraction process may be carried out to any degree, but is usually continued to exhaustion. Typical yields (=extract dry matter, based on the quantity of raw material used) in the extraction of the litchi shells are of the order of 2 to 3%, based on OPC A2.

### Chromatography and liquid/liquid extraction

The chromatographic purification and the liquid/liquid extraction may be carried out in known manner. Resins without any functional groups have proved to be a suitable material for the chromatography column. The separation is preferably carried out at 15 to 30° C., the mobile solvent being selected, above all, from lower aliphatic alcohols containing 1 to 4 carbon atoms, more particularly methanol or ethanol. Water-immiscible solvents, such as butanol or ethyl acetate for example, have proved to be useful for the following extraction step which is preferably carried out at temperature of at least 25° C., the upper limit being determined by the boiling point of the solvent.

### Commercial Applications

Extracts of Litchi sinensis in general and the OPC A2-enriched new extracts in particular inhibit MMPs to a greater extent than known products and are suitable, for example under the heading of “cosmetic inside”, for the production of food supplements. In one particular application form, the extracts are used in encapsulated form, for example as gelatin capsules, or in microencapsulated form. Suitable microcapsules with diameters of 0.0001 to 5 mm and processes for their production are described, for example, in WO 01/01926, WO 01/01927, WO 01/01928 and WO 01/01929 (Primacare).

The present invention also relates to the use of the new OPC A2-rich extracts for the production of cosmetic and/or pharmaceutical preparations in which they may be present in quantities of 0.1 to 10, preferably 0.5 to 5 and more particularly 1 to 2% by weight.

### EXAMPLES

### Example 1

3 kg size-reduced shells of Litchi sinensis with an OPC A2 content of 0.4% by weight were introduced into a 50-liter reactor and extracted with 30 kg aqueous methanol for 60 mins. at 50° C. 23 kg of a liquid with a solids content of 500 g were obtained. The liquid had an OPC A2 content of 2% by weight. After concentration of the extract to a volume of 1.5 kg, the liquid was subjected to chromatographic purification at 25° C. in a column with a coating free from functional groups using ethanol as the mobile solvent. 2.5 kg extract with a solids content of 80 g were obtained. The extract was re-concentrated to a volume of 500 g and then subjected at 45° C. to liquid/liquid chromatography with aqueous butanol. Whereas the intensively red-colored aqueous phase only contained less than 1% by weight OPC A2, based on the active substance content, 50 g of a light reddish colored organic phase were obtained which had an OPC A2 content of 23% by weight, based on the active substance content.

### Comparison Example C1

As in Example 1, 3 kg size-reduced shells of Litchi sinensis with an OPC A2 content of 0.4% by weight were introduced into a 50-liter reactor and extracted with 30 kg aqueous methanol for 60 mins. at 50° C. After removal of the methanol, a residue of 500 g was obtained and was taken up in 1.5 litres distilled water. This solution was then repeatedly extracted with a total of 3 liters ethyl acetate. After separation, a light reddish colored organic phase was obtained which had an OPC A2 content of 10% by weight, based on the active substance content.

### Effectiveness against proteases

During any inflammation, skin proteases, such as collagenase for example, are release from the polymorphonuclear neutrophilic granulocytes or macrophages. A similar process takes place in the skin of elderly people under the influence of UV rays. As already mentioned, the proteases—also known as matrix metalloproteases (MMPS) by virtue of their content of central zinc ions—catalyze the fragmentation of connective tissue proteins. The test substances were studied for collagenase inhibition using bacterial collagenase (Clostridium histolyticum) on gelatin as a natural nutrient medium which had been marked with fluorochromium (FITC, Calbiochem). The incubation time was 60 mins. at 20° C. and the hydrolysis of the substrate was monitored via the fluorescence at 393 nm (excitation at 328 nm). The results are set out in Table 1. The collagenase inhibition is expressed in %.

TABLE 1
Collagenase inhibition (in %-rel.)

| | | Concentration % (w/v) | | |
|---|---|---|---|---|
| Example | Test product | 0.001 | 0.005 | 0.01 |
| 2 | Product of Example 1 | 18 | 46 | 67 |
| C2 | Product of Example C1 | 11 | 27 | 34 |

The results show that the test substances according to the invention have a significant inhibiting effect as a function of their concentration.

1-10. (canceled)

11. A botanic extract of Litchi sinensis comprising, based on active substance content, at least 15% by weight of an oligomeric proanthocyanidin of the OPC A2 type.

12. The botanic extract of claim 11 wherein the oligomeric proanthocyanidin of the OPC A2 type is present in an amount by weight of from 20% to 25%.

13. A process of obtaining at least 15% by weight, based on active substance content, of an oligomeric proanthocyanidin of the OPC A2 type from a botanic extract of Litchi sinensis, which process comprises:
(a) extracting shells of Litchi sinensis with a lower aqueous aliphatic alcohol solvent;
(b) subjecting the extract to a chromatographic separation, which separation forms an OPC A2-rich fraction;
(c) subjecting the OPC A2-rich fraction to a liquid/liquid solvent extraction, which solvent extraction forms an aqueous phase and a water-immiscible phase; and
(d) recovering the OPC A2-rich fraction of at least 15% by weight from the water-immiscible phase.

14. The process of claim 13 wherein from 20% to 25% by weight of the OPC A2 type is recovered.

15. The process of claim 13 wherein the lower aqueous aliphatic alcohol solvent in (a) contains methanol or ethanol, or a mixture thereof.

16. The process of claim 13 wherein (a) is carried out at a temperature of from 20 to 100° C.

17. The process of claim 16 wherein (a) is carried out at a temperature of from 50 to 70° C.

18. The process of claim 13 wherein (a) is carried out in the presence of an inert gas.

19. The process of claim 13 wherein (b) is carried out in the presence of a solvent selected from the group consisting of lower aliphatic alcohols containing from 1 to 4 carbon atoms, or mixtures thereof.

20. The process of claim 13 wherein (c) is carried out in the presence of a water-immiscible solvent.

21. A composition for oral or cosmetic administration which comprises an extract of Litchi sinensis containing an effective amount by weight of an oligomeric proanthocyanidin of the OPC A2 type for inhibiting matrix metalloprotease activity and a carrier suitable for oral or cosmetic administration.
